(19) United States
(12) Patent Application Publication
(10) Pub. No.: US 2009/0240344 A1
(43) Pub. Date: Sep. 24, 2009

(54) MULTI-LAYER POLYMERIC PROSTHETIC LINER

(75) Inventors: James M. Colvin, Hilliard, OH (US); Christopher T. Kelley, Grandview Heights, OH (US); Mark A. Groves, Columbus, OH (US); Jeffrey A. Denune, Galloway, OH (US)

Correspondence Address: STANDLEY LAW GROUP LLP, 6300 Riverside Drive, Dublin, OH, 43017, US

(73) Assignee: THE OHIO WILLOW WOOD COMPANY, Mount Sterling, OH (US)

(21) Appl. No.: 12/407,362

(22) Filed: Mar. 19, 2009

Related U.S. Application Data
(60) Provisional application No. 61/037,765, filed on Mar. 19, 2008.

(51) Int. Cl. A61F 2/80

(52) U.S. Cl. 623/36

(57) ABSTRACT

A multi-layer prosthetic liner having an inner polymeric material layer surrounded by an outer polymeric material layer. The outer polymeric material layer is dissimilar to the inner polymeric material layer. A tying layer may reside between the inner polymeric material layer and the outer polymeric material layer. Any embodiment of the liner may be wholly or partially covered on its exterior with a material that exhibits low-friction and/or wicking characteristics.

### CROSS-REFERENCE TO RELATED APPLICATION

This application claims priority to U.S. Provisional Patent Application Ser. No. 61/037,765, filed Mar. 19, 2008, titled MULTI-LAYER POLYMERIC PROSTHETIC LINER, which is incorporated herein by reference.

### BACKGROUND

The present invention is directed to a prosthetic liner designed to function as an interface between an amputee's residual limb and the interior of a prosthetic socket. More particularly, the present invention is directed to such a prosthetic liner having a multi-layer polymeric construction.

In recent years, polymeric prosthetic liners (“liners”) have gained tremendous favor among amputees due to the comfort, security of suspension, protection of the residual limb, and ease of use associated therewith. Amputees commonly employ a liner as a stand-alone interface between their residual limb and the interior of a prosthetic socket. Such a liner may optionally include a docking element for mechanical attachment of the liner to a prosthesis. Liners can be of standard “off-the-shelf” design, or may be custom designed for a particular amputee.

Liners may be comprised of various polymeric materials, including silicone, urethane, or thermoplastic elastomer (TPE) gels. Silicone and urethane are thermosetting materials. The thermoset materials tend to be harder than the TPE gels. For example, existing silicone liners typically have a hardness of between about 35-65 on the Shore 00 scale. While TPE gels may be formulated to a wide hardness range, it has been found that TPE gels having a hardness of between about 15-35 on the Shore 00 scale provide optimal comfort for most users.

While harder thermoset materials generally exhibit higher durability than TPE gel materials, thermoset materials also typically offer less comfort. Conversely, while softer TPE gel materials offer higher levels of comfort, TPE gel materials are typically less durable than thermoset materials. Liners made from thermoset materials and liners made from TPE gels have both found success in the field of prosthetics but, unfortunately, users must often choose between liners of high durability and liners of high comfort. Consequently, it can be understood that there is a need for a prosthetic liner that exhibits high levels of both durability and comfort.

TPE gels tend to flow and change shape when exposed to sufficient heat and/or pressure. This TPE gel characteristic can be beneficial to an amputee by providing relief when a particular portion(s) of an amputee's residual limb is subjected to high contact forces within a prosthetic socket. This may occur, for example, where the interior of the prosthetic socket presses against a bony prominence of the residual limb.

Conversely, the tendency of TPE gels to change shape can also be problematic if the result is an unacceptably large change in the overall shape of a liner manufactured from such a gel. For example, a change in the shape of a TPE gel due to heat and/or pressure may cause an unacceptable expansion of a liner along the edge of its open (proximal) end. In such a case, the liner may tend to roll down or not feel secure. Thus, there is also a need for a TPE gel liner that permits at least some change in the shape of the gel while simultaneously limiting the resulting deformation of a liner manufactured therefrom.

At least certain known liners are manufactured from a TPE gel that is infused with mineral oil. The mineral oil may be used to control the hardness of the TPE gel and may also function to condition the skin of a residual limb during liner use. As would be understood by one having ordinary skill in the art, the mineral oil leaches from the TPE gel while the liner is donned, helping to condition the skin of the residual limb. With respect to TPE gel liners having a fabric outer covering, it has been discovered that mineral oil can also permeate through the fabric over time. Such a loss of mineral oil can eventually reduce the thickness of the liner and shorten its useful life. Thus, it can be understood that there is also a need for a fabric-covered TPE gel liner that eliminates or minimizes the transfer of mineral oil through the outer cover.

There has also been recent increased interest in vacuum-based prosthesis suspension. This suspension technique relies on evacuation of air from the prosthetic socket into which a liner-covered residual limb has been inserted. The elevated vacuum produces a drawing effect on the residual limb that tends to keep it securely retained in the prosthetic socket. Unfortunately, the use of elevated vacuum can also exert additional forces on a liner, which forces can negatively affect its performance and useful life. For instance, it has been found that the mineral oil in TPE gel liners can be drawn out of the gel when exposed to vacuum, potentially shortening the life of the liner and possibly harming the associated vacuum device. Under sufficient loading, it may even be possible to pull small pieces of a TPE gel through the fabric outer covering. Consequently, as can be understood, there is also a need for a TPE gel liner that is not adversely affected when used with a vacuum suspension system.

### SUMMARY OF THE OF THE GENERAL INVENTIVE CONCEPT

A liner of the present invention is designed to enclose at least a portion of a residual limb. As such, a liner of the present invention generally includes an open end for allowing introduction of the residual limb, and a closed end opposite the open end. The closed end generally abuts and cushions the distal end of the residual limb when the liner is donned. Such a liner may be used by an upper or lower extremity amputee.

A liner of the present invention is of multi-layer construction. More specifically, a liner of the present invention generally includes an inner polymeric layer designed to provide comfort and an outer layer designed to provide durability, the outer layer being of a material that is dissimilar to the material of the inner layer. For example, a liner of the present invention may be comprised of an inner layer of a TPE gel, such as a TPE gel described in one or more patents to Bruce G. Kania. The inner layer may also be comprised of another suitably soft material such as, for example, TPE foam, silicone foam, etc.

To this end, the outer layer of a liner of the present invention is preferably comprised of a polymeric material that exhibits mechanical properties superior to those of the material of an associated inner layer. For example, and without limitation, the polymeric outer layer of a liner of the present invention may be comprised of a material that exhibits superior creep resistance, abrasion resistance, puncture resistance, and/or hardness than the polymeric material of an inner layer of the liner. In certain embodiments, the outer layer of a liner of the present invention is preferably comprised of a material that is capable of preventing or minimizing the transfer of mineral oil through the outer layer.

For purposes of comparison, the mechanical properties of an inner and outer polymeric layer of a liner of the present invention may be measured by various methods. For example, abrasion resistance may be measured using the ASTM D3884-01 standard for testing the Abrasion Resistance of Textile Fabrics (Rotary Platform, Double-Head Method). This test method is performed by abrading a polymer surface for 1,000 cycles and then measuring the loss in weight. During one comparative test performed in this manner, it was found that a particular TPE gel of interest experienced 0.75 g of weight loss, whereas particular silicones of interest (i.e., Wacker Chemie AG Elastosil 4110, and Quantum Silicones Specialties 92-73-1A and 93-73-1B) experienced only 0.15 g of weight loss. Puncture resistance may similarly be measured by various methods, including pushing a probe through a uniform thickness of a particular polymer of interest. During one comparative test performed in this manner, a particular TPE gel of interest punctured at approximately 5.73 lbf. at 5.4 in. elongation, whereas particular silicones of interest (i.e., Wacker Chemie AG Elastosil 4110, and Quantum Silicones Specialties 92-73-1A and 93-73-1B) did not puncture until 49.65 lbf. was exerted on the probe with the material sample elongated at 4.438 in.

For example, acceptable thermoset outer layer materials may include silicone and polyurethane. Examples of potentially useable thermoplastic outer layer materials include TPEs with reduced amounts of mineral oil, higher molecular weight TPEs, and TPEs containing reinforcing additives such as, without limitation, Kevlar or nylon in the form of, for example, pulped fibers, flocked fibers, short and long fiber strands, powders and nano-sized particles (e.g., nano-clay, nano-tubes, and graphene plates).

Regardless of the particular materials used to form the inner and outer layers of a liner of the present invention, it is preferred that each layer exhibit an acceptable hardness range. As a result of such a construction, a liner of the present invention will generally provide enhanced comfort and cushioning, increased durability, and improved liner shape retention in comparison to known prosthetic liners.

For various reasons, it is generally beneficial to provide a liner of the present invention with an exterior surface that exhibits low friction and/or that allows for wicking. For example, a low-friction exterior surface facilitates donning of a liner by inversion and rolling onto the residual limb. That is, a low-friction surface allows a liner to be turned inside-out and subsequently rolled onto a residual limb without the polymeric portion thereof sticking to itself. Further, and as can be easily understood, a low-friction exterior surface facilitates insertion of a liner-covered residual limb into a prosthetic socket.

The provision of an exterior surface that facilitates wicking may be beneficial for several reasons. For example, when a liner of the present invention is used with a suction (vacuum) suspension system, providing the liner with an exterior surface that facilitates wicking of the air as it is evacuated from within the prosthetic socket of an associated prosthesis allows the vacuum to draw on substantially the entire exterior surface of the liner that is covered by the wicking surface within the socket. Consequently, the weight of a suspended prosthesis can be more evenly distributed over the surface of an amputee's residual limb.

To this/these end(s), any embodiment of a liner of the present invention may be partially or wholly covered with one or more materials that exhibit low friction and/or wicking characteristics. Such materials may include various fabrics, fibers or films. Such a material, or some combination of such materials, may be made to form the exterior surface of a liner of the present invention by a variety of techniques. For example, one or more acceptable low-friction and/or wicking materials may be affixed to a subjacent polymeric material layer during molding of the liner (i.e., the polymeric material may be molded directly to the low-friction and/or wicking material). Alternatively, one or more of such materials may be affixed to a subjacent polymeric material layer with an adhesive or other chemical bonding agent, a cast elastomer film, etc.

Alternatively, another type of coating could be applied to the exterior of the liner to reduce friction, such as may be accomplished by corona or plasma treating or by Parylene coating. Further, a chemical additive could be added to the outer polymeric layer (such as during initial mixing) to result in a matte surface finish that will reduce friction. One exemplary chemical additive that may be used in this manner with respect to silicone materials is QSil Matting Agent, produce by Quantum Silicones. Ion Implantation techniques (see, e.g., U.S. Pat. No. 5,223,309) may also be employed to reduce the coefficient of friction exhibited by an outer polymeric layer material of a liner of the present invention. Still further, fluorine gas treatment may be employed to reduce the coefficient of friction exhibited by an outer polymeric layer material of a liner of the present invention. Also, textures, labyrinth paths, embossed features, de-bossed features, a combination of embossed and de-bossed features, radiating fins and/or channels could be selectively molded into the outside surface of a liner of the present invention (and/or the inside surface of an associated prosthetic socket) to permit wicking over appropriate areas of the liner in lieu of using fabric or other wicking type materials.

The general benefits of a low-friction exterior liner surface notwithstanding, in certain circumstances it may also be advantageous to design a liner of the present invention with one or more exterior surface portions that exhibit high friction. As described in more detail below, such areas of high friction may be provided at various locations along the exterior surface of a liner. Further, more than one area of a liner exterior surface may be made to exhibit high friction.

A liner of the present invention may be of “off-the-shelf” (generic) design, as would be understood by one skilled in the art to mean that the liner is not custom fit to each amputee but, rather, is of a substantially generic shape (as shown, for example, in the drawing figures). Typically, but not exclusively, this generic shape is substantially frusto-conical. Such a generic liner may be available in several sizes so as to better accommodate a large number of amputees without requiring the use of a custom manufacturing process. A generic liner may also be of locking or non-locking design, the latter embodiment being generally referred to in the art simply as a cushion liner. A locking liner would be understood to include an attachment element at a distal end thereof that is adapted to connect the liner to a retention mechanism associated with a prosthetic socket. Typically, but not exclusively, the attachment element is adapted to retain a pin or a lanyard that releasably engages a locking mechanism located at the distal end of a prosthetic socket.

Alternatively, a liner of the present invention may be custom molded for a particular amputee. In contrast to a generic liner, a custom liner is designed and manufactured specifically to fit the residual limb of an amputee. A custom liner design and/or molding technique that may be used for this purpose is substantially disclosed in U.S. Pat. No. 7,162,322, although other custom liner design and/or manufacturing methods may also be employed.

Regardless of whether a liner of the present invention is of generic or custom design, a number of optional features may be offered and provided. For example, a liner of either type may be offered in a number of different thicknesses, which thicknesses may be uniform or varying in nature. For example, any of the thickness profiles described and/or shown in U.S. Pat. No. 6,964,688 may be employed. The overall thickness of a liner of the present invention may vary but should be sufficient to allow the liner to function as a standalone interface between an amputee's residual limb and the interior of a prosthetic socket.

In the case of a custom liner, a number of options may be offered to an amputee. Any of the liner options described and/or shown in U.S. Pat. No. 7,162,322 may be provided on/in a liner of the present invention. For example, such options may include, without limitation: specification of polymeric material thickness, whether overall or only as to select areas; specification of the amount of compression exerted by a liner on a residual limb, whether overall or only as to select areas (e.g., by varying the circumference of the liner, gel properties, and/or fabric properties); selection of exterior surface covering properties (e.g., elasticity, wear, color, thread type and/or thread color, reinforcement type and/or position, etc.); selection of suspension mechanism (e.g., type, size, position, etc.); inclusion of bladders for volume adjustment (e.g., number, size, position, built-in, attached, etc.); specification of polymeric material properties (e.g., durometer, elasticity, additives, etc.); inclusion/specification of sensors for providing feedback to an amputee or practitioner or for controlling a prosthesis (e.g., type, position, built-in, attached); inclusion of fabric over select areas of the interior surface of the liner (e.g. for reinforcement or comfort); and/or inclusion of a distal attachment element.

A better understanding of a liner of the present invention can be gained by review of the following description of several exemplary embodiments thereof, along with the associated accompanying drawing figures.

### BRIEF DESCRIPTION OF THE DRAWINGS

In addition to the features mentioned above, other aspects of the present invention will be readily apparent from the following descriptions of the drawings and exemplary embodiments, wherein like reference numerals across the several views refer to identical or equivalent features, and wherein:

FIG. 1a depicts one exemplary embodiment of a multi-layer prosthetic liner of the present invention;

FIG. 1b is a cross-sectional view taken along line 1b-1b of the liner of FIG. 1a;

FIG. 2a illustrates another exemplary embodiment of a multi-layer prosthetic liner of the present invention;

FIG. 2b is a cross-sectional view taken along line 2b-2b of the liner of FIG. 2a;

FIG. 3a shows another exemplary embodiment of a multi-layer prosthetic liner of the present invention, wherein a tying feature is present between two polymeric material layers;

FIG. 3b is a cross-sectional view taken along line 3b-3b of the liner of FIG. 3a;

FIGS. 4a-4b depict an exemplary multi-layer prosthetic liner of the present invention, the liner having an exterior surface that is fully covered with a low-friction and/or wicking material;

FIG. 4c is a front elevation view of the exemplary liner of FIGS. 4a-4b, wherein the presence of the tying layer and exterior surface material can be more readily observed;

FIG. 4d is a cross-sectional view taken along line 4d-4d of the liner of FIG. 4c;

FIGS. 5a-5b illustrate an exemplary multi-layer prosthetic liner of the present invention, the liner having an exterior surface that is partially covered with a low-friction and/or wicking material;

FIGS. 6a-6b illustrate an alternative embodiment of an exemplary multi-layer prosthetic liner of the present invention having an exterior surface that is partially covered with a low-friction and/or wicking material; and

FIGS. 7a-7c illustrates an alternative embodiment of an exemplary multi-layer prosthetic liner of the present invention having an exterior surface with molded wicking channels.

### DETAILED DESCRIPTION OF THE EXEMPLARY EMBODIMENT(S)

Several exemplary embodiments of a prosthetic liner of the present invention are provided below. These exemplary embodiments are provided solely for the purpose of illustration, and not limitation. As described above, each embodiment includes inner and outer layers of polymeric materials of dissimilar mechanical properties. With respect to the particular exemplary embodiments described below, each embodiment includes an outer polymeric material layer that is harder than the inner polymeric material layer that it surrounds. It should be realized, however, that such a dissimilarity in hardness characteristics is not an essential feature of a liner of the present invention.

Any of the exemplary polymeric materials described above may comprise the respective inner and outer layers of the disclosed exemplary embodiments. For example, any of the exemplary liner embodiments may include an inner layer of a thermoplastic elastomer (TPE) gel or another suitably soft material such as a TPE foam, silicone foam, etc. The TPE gel may be a block copolymer material. Similarly, any of the exemplary liner embodiments may include an outer layer comprised of a more durable thermoset or thermoplastic material. For example, acceptable thermoset outer layer materials may include silicone (e.g., Wacker Chemie AG Elastosil 4110, and Quantum Silicones Specialties 92-73-1A and 93-73-1B) and polyurethane (e.g., BJB Enterprises, Inc., TC 5101A and 5101B). Examples of potentially useable thermoplastic outer layer materials include TPEs with reduced amounts of mineral oil, higher molecular weight TPEs, and TPEs containing reinforcing additives such as, without limitation, pulped fibers, flocked fibers, short and long fiber strands, powders and nano-sized particles (e.g., nano-clay, nano-tubes, and graphene plates). It is also contemplated that an outer layer of a liner of the present invention may be comprised of a thermoplastic urethane (TPU) or a thermoplastic silicone-urethane copolymer (TSPU). Examples of acceptable TSPUs include, without limitation, PurSil AL-5 and AL-10. It is also contemplated that an outer layer of a liner of the present invention may be comprised of a shear thickening or thinning material or fluid. Micro balloons and/or micro spheres could be added to the inner layer and/or the outer layer to reduce the overall weight of the liner.

Regardless of the particular polymeric material comprising the inner and outer layers of a liner of the present invention, it is preferred that the material of each layer have a hardness that falls within a desired range. For example, it is preferred, but not essential, that an inner layer of a liner of the present invention exhibit a hardness of between about 10-40 and, more preferably, between about 20-35, both on the Shore 00 scale. In contrast, it is preferred, but not essential, that the outer layer of a liner of the present invention exhibit a hardness of between about 40-70 on the Shore 00 scale. Consequently, any of the disclosed exemplary liner embodiments may employ polymeric materials of the aforementioned types and/or hardness ranges.

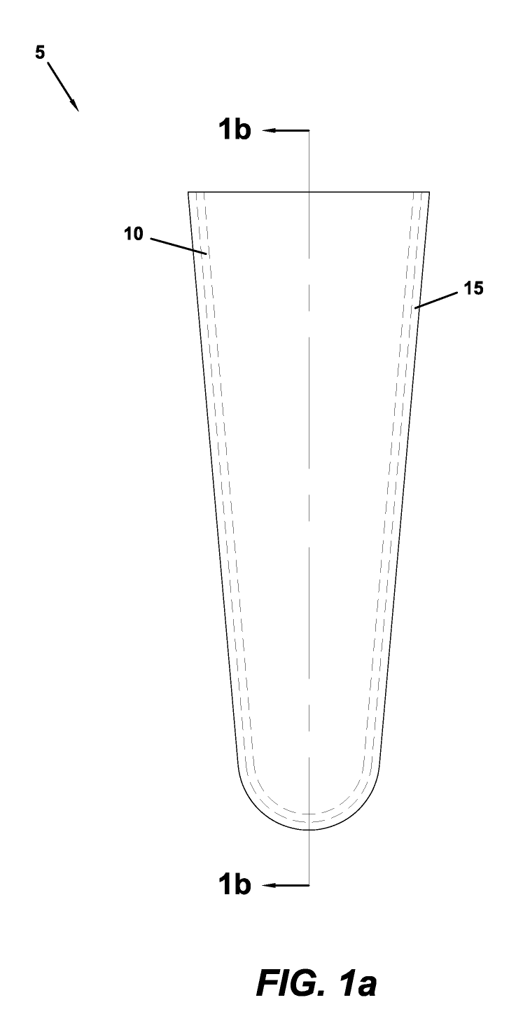

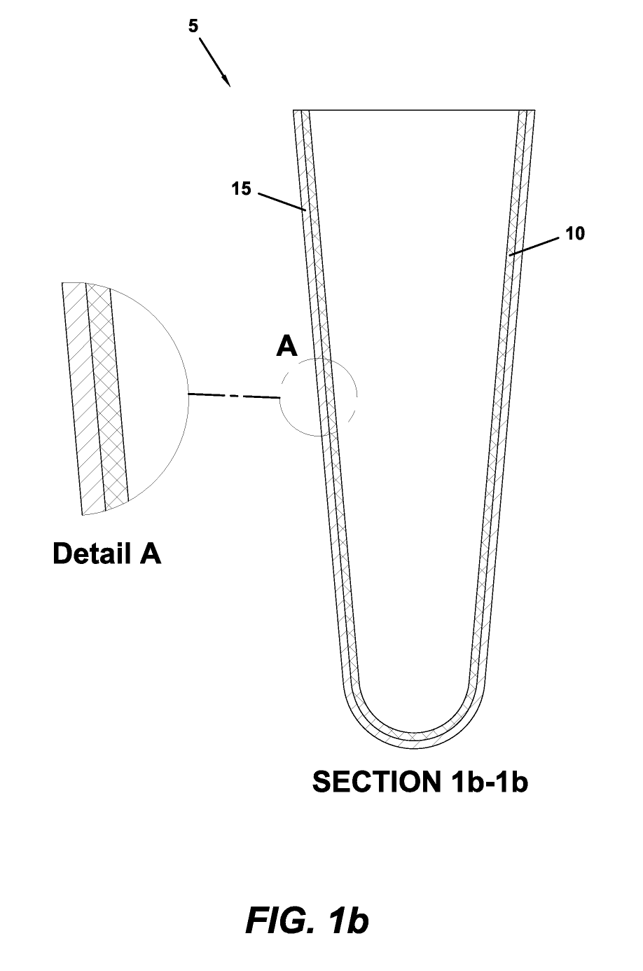

One exemplary embodiment of a multi-layer prosthetic liner (“prosthetic liner” or “liner”) 5 of the present invention is illustrated in FIGS. 1a-1b. As shown, the liner 5 includes a softer inner polymeric material layer 10 and a harder outer polymeric material layer 15. The inner layer 10 of the liner will abut the skin of a residual limb when donned, while the outer layer 15 will abut the interior wall of a prosthetic socket. The inner and outer layers may be connected (joined) by any of the techniques, or combination of techniques, mentioned herein.

In this particular embodiment of the liner 5, the soft inner polymeric material layer 10 and the harder outer polymeric material layer 15 could be chemically bonded at the interface between the hard and soft layer during the manufacturing process by overmolding. Various techniques can be used to enhance the bond created by overmolding including, but not limited to, using a graft copolymer in one or both of the layers to provide compatible bond sites at the interface surface, and increasing the surface energy of the primary layer 10 using corona, plasma or flame etching, fluorine gas treatments or various primers so that the secondary layer 15 will bond with the primary layer during the overmolding process.

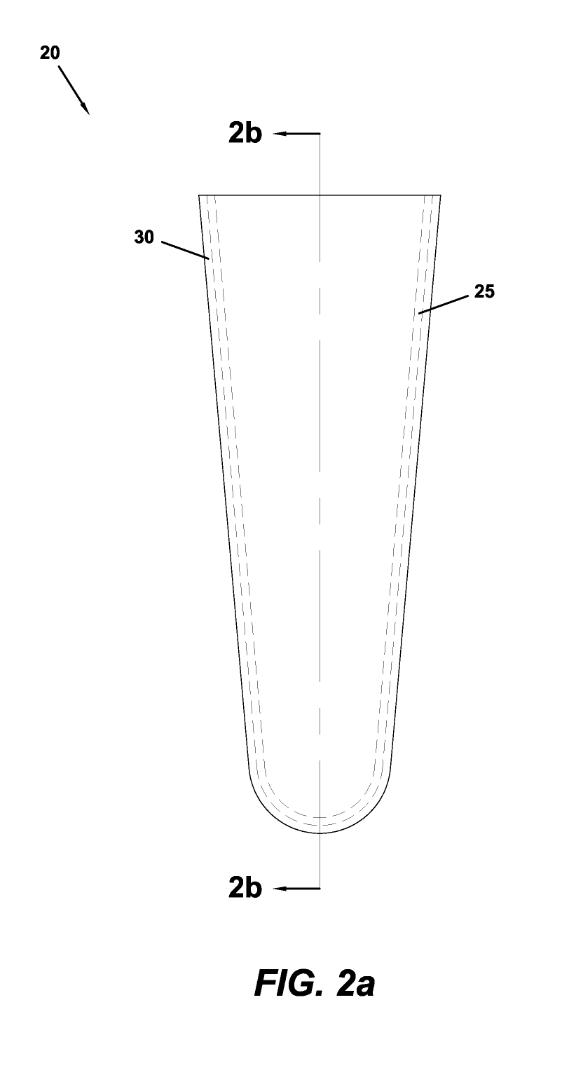

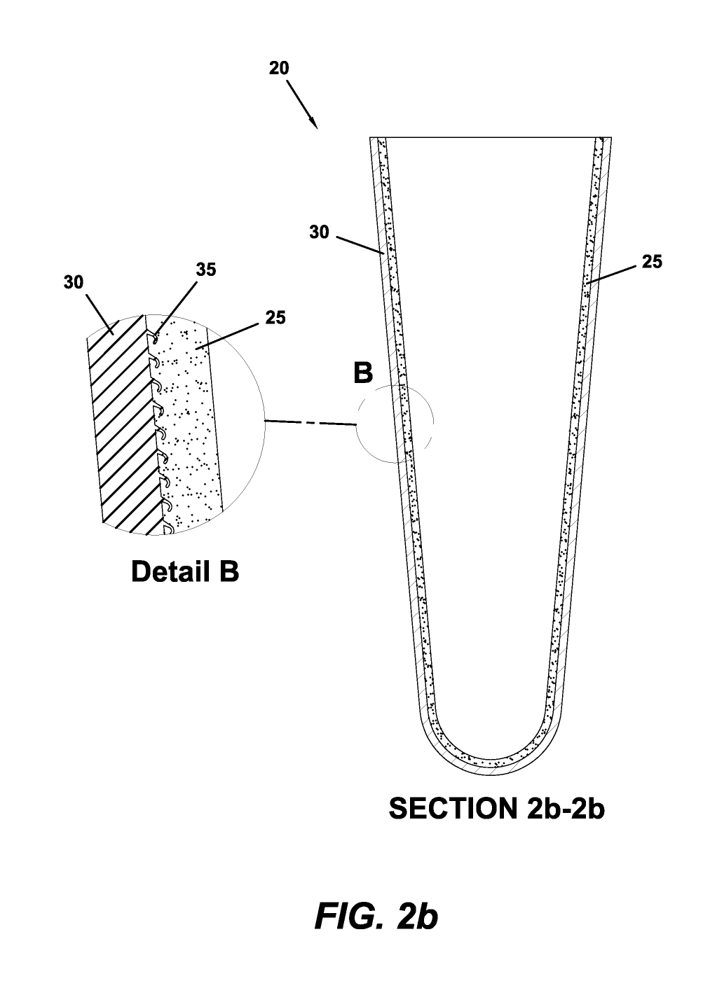

Another exemplary embodiment of a multi-layer prosthetic liner (“prosthetic liner” or “liner”) 20 of the present invention is illustrated in FIGS. 2a-2b. As shown, the liner 20 includes a softer inner polymeric material layer 25 and a harder outer polymeric material layer 30. The inner layer 25 of the liner will abut the skin of a residual limb when donned, while the outer layer 30 will abut the interior wall of a prosthetic socket.

As can be best observed in Detail B of FIG. 2b, this particular embodiment of the liner 20 includes an interlocking feature between its inner and outer polymeric material layers 25, 30. While it should be realized that a number of interlocking designs could be employed for this purpose, this particular embodiment of a liner 20 of the present invention includes a multitude of small hooks 35 for joining the inner and outer layers 25, 30 of the liner. The hooks 35 (or other interlocking features) may be molded into and extend outward from the outer polymeric layer 30 toward the inner polymeric layer 25. For example, the outer layer 30 of the liner 20 may be molded first, followed by molding thereto of the inner polymeric layer 25.

It may also be possible to reverse this exemplary design and molding technique, such that the hooks 35 extend from the inner polymeric material layer 25 instead of the outer polymeric material layer 30. Further, sections of separately molded or otherwise-produced hooks may instead be bonded or otherwise affixed to a polymeric material layer for the same purpose. By whatever technique employed, hooks may be made to cover substantially all, or only a certain portion(s) of a polymeric material layer of a liner of the present invention.

The presence of the hooks 35 provides for increased adhesion between the inner and outer polymeric material layers 25, 30. Use of the hooks 35 of this embodiment may be augmented by any of the other techniques, or combination of techniques, mentioned above for joining inner and outer polymeric layers.

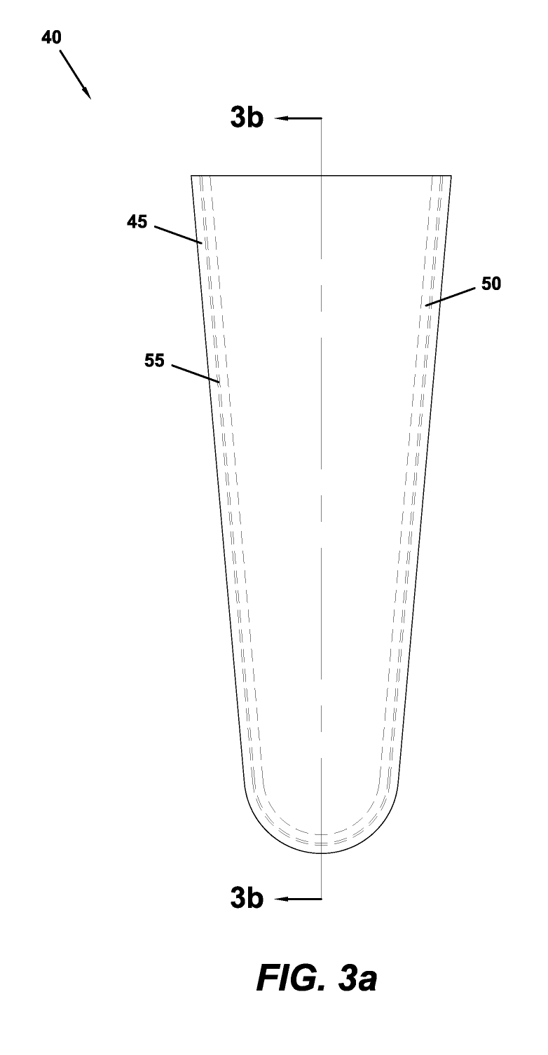

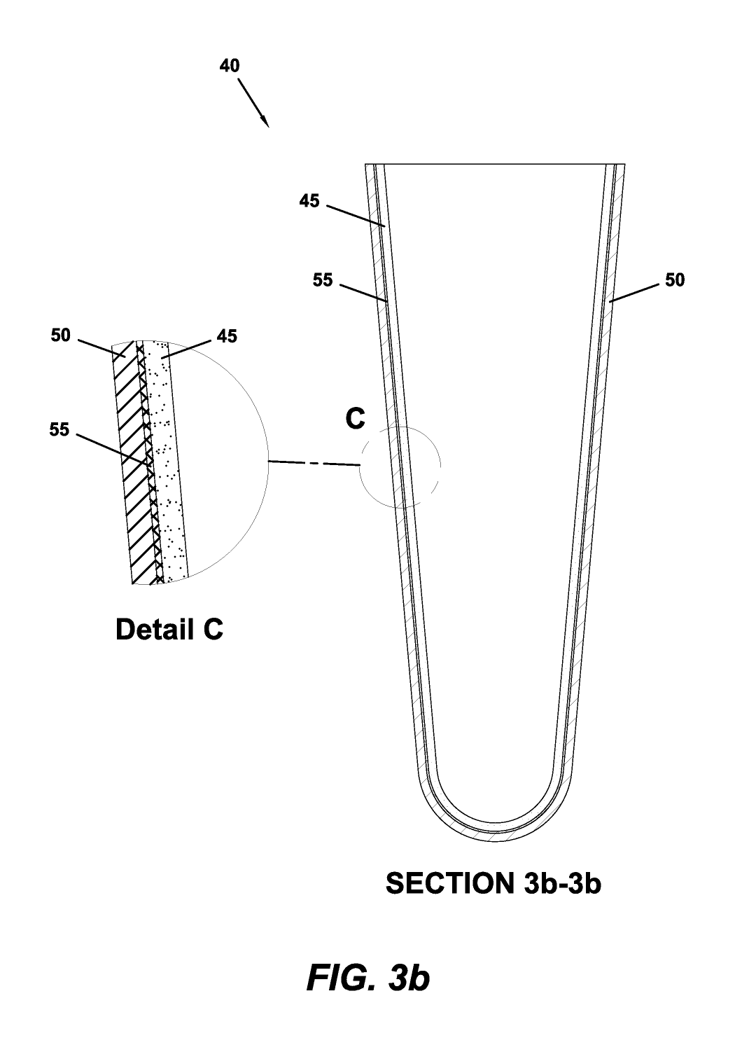

Another exemplary embodiment of a multi-layer prosthetic liner 40 of the present invention is illustrated in FIGS. 3a-3b. As shown, the liner 40 again includes a softer inner polymeric material layer 45 and a harder outer polymeric material layer 50. The inner layer 45 of the liner will abut the skin of a residual limb when donned, while the outer layer 50 will abut the interior wall of a prosthetic socket.

Unlike the exemplary liner 5 of FIGS. 1a-1b and the liner 20 of FIGS. 2a-2b, however, this liner embodiment includes a tying layer 55 that resides between its inner and outer polymeric material layers 45, 50. It has been discovered that the materials selected for the manufacture of the inner and outer layers of a liner of the present invention may resist bonding due to the dissimilar nature thereof. Consequently, it may be beneficial to locate an intermediary (tying) layer between the inner and outer polymeric material layers of at least certain embodiments of a liner of the present invention.

A tying layer for use in the present invention may be comprised of a material that facilitates bonding between the actual inner and outer polymeric material layers of a liner. For example, such a tying layer may comprise a thin film that initiates a bond-resulting chemical reaction between the polymeric material layers when subjected to heat or some other triggering mechanism. Alternatively, and as described above, the tying layer may instead be simply a coating or thin film of an adhesive material that is able to bond to the selected polymeric materials between which it is arranged.

In other embodiments of the present invention, and as shown in the exemplary embodiment of FIGS. 3a-3b, the tying layer 55 may be comprised of a fabric or similar material. Various fabrics may function acceptably well for this purpose. Obviously, a selected tying layer fabric should bond well to each of the associated inner and outer layers of polymeric material. Preferably, but not essentially, suitable tying layer fabrics will also be thin, exhibit substantial elongation characteristics, and present a large surface area to which the polymeric materials can bond. A fiber material in the form of flocking or particulates may also serve as a suitable tying layer. Other suitable tying materials of such a nature may include stitched polyurethane (or other elastomeric) film material (as available from, e.g., Xymid) and flocked TPE film (as available from e.g., Lextra).

As described above, any embodiment of a liner of the present invention may have an outer surface that is comprised wholly or partially of a low-friction and/or wicking-capable material. Such materials may include various fabrics, fibers or films. Such a material, or a combination of such materials, may be made to form the exterior surface of a liner of the present invention by a variety of techniques. For example, one or more acceptable low-friction and/or wicking-capable materials may be molded directly to a subjacent polymeric material layer during the liner manufacturing process. Alternatively, one or more of such materials may be affixed to a subjacent polymeric material layer with an adhesive or other chemical bonding agent, a cast elastomer film, etc. An acceptable cast elastomer film process is described in U.S. Pat. No. 6,626,952 and/or U.S. Pat. No. 7,001,563.

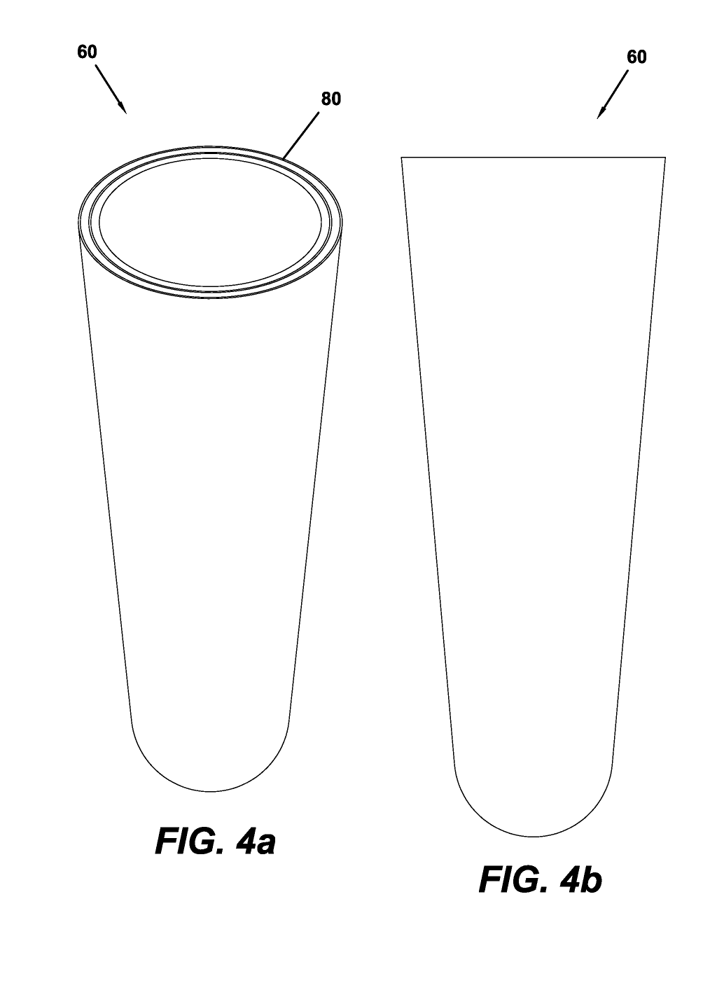

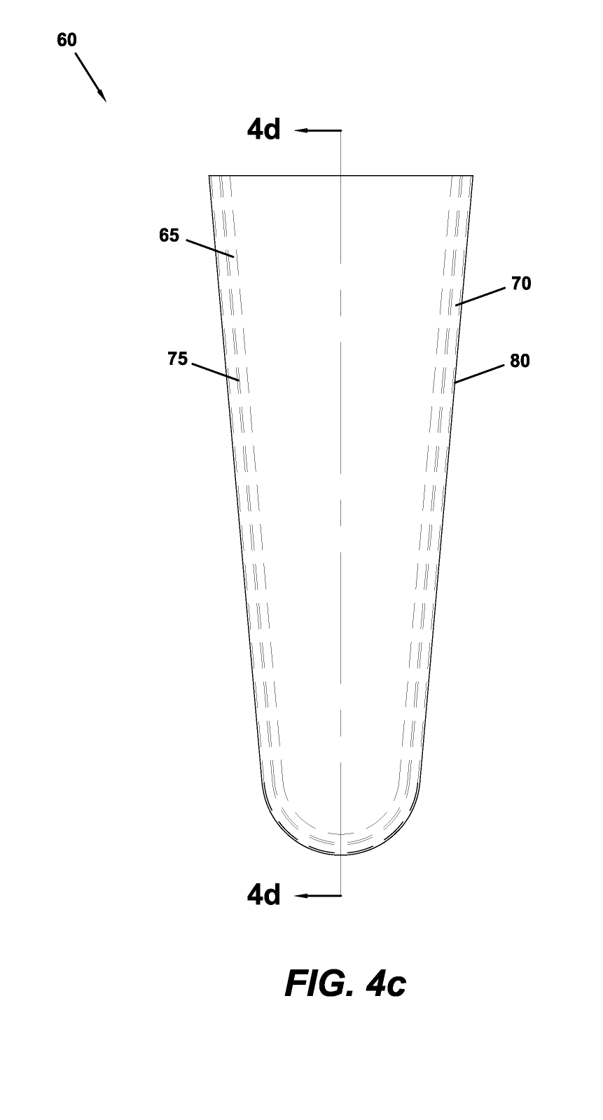

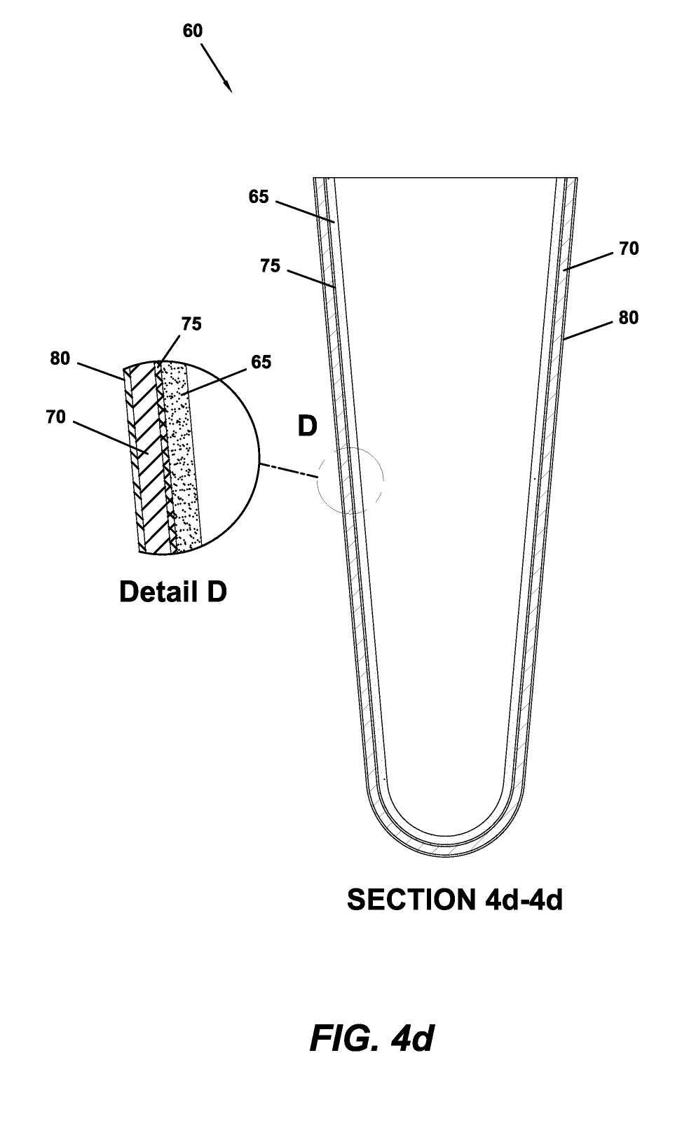

One embodiment of such a liner is exemplified by the fully fabric-covered prosthetic liner 60 of FIGS. 4a-4d. While any contemplated liner embodiment of the present invention may include such an exterior covering, the particular liner construction shown in FIGS. 4a-4d includes an intermediate tying layer like that of the liner 40 of FIGS. 3a-3b. Consequently, this particular liner 60 includes a softer inner polymeric material layer 65 that will abut the skin of a residual limb when donned, the inner polymeric layer being surrounded by a harder outer polymeric material layer 70. A fabric tying layer 75 resides between the inner and outer polymeric layers 65, 70 of the liner 60.

Unlike the liner 40 of FIGS. 3a-3b, this exemplary liner 60 further includes a fabric exterior surface (covering) 80. The fabric exterior covering 80 may be affixed to the outer polymeric material layer 70 by any technique known in the art including, but not limited to, the methods described above. As shown, the fabric exterior covering 80 envelops substantially the entire exterior surface of the outer polymeric material layer 70. The fabric exterior covering 80 preferably exhibits one or more of the characteristics enumerated above.

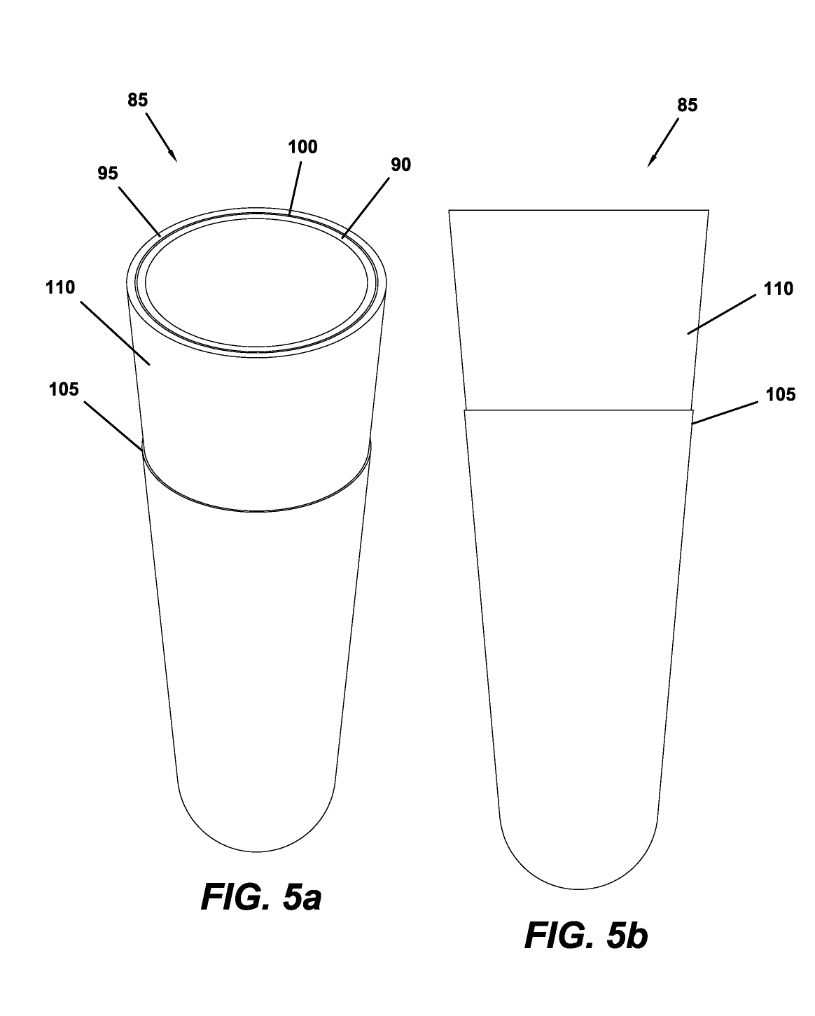

An alternate embodiment of a liner 85 of the present invention is illustrated in FIGS. 5a-5b. As with the liner of FIGS. 4a-4d, this particular liner 85 includes a softer inner polymeric material layer 90 that will abut the skin of a residual limb when donned, the inner polymeric layer being surrounded by a harder outer polymeric material layer 95 that is separated therefrom by a fabric tying layer 100. Unlike the liner of FIGS. 4a-4d, however, the outer surface of this exemplary liner 85 includes a fabric exterior covering 105 that extends over less than the entire exterior surface of the outer polymeric material layer 95.

As shown in FIGS. 5a-5b, the fabric exterior covering 105 of the liner 85 extends from the distal (closed) end of the liner toward the proximal (open) end, but terminates prior thereto. As such, a portion 110 of the outer polymeric material layer 95 of this particular liner 85 remains exposed in the form of circumferential band along the open end of the liner. Such a configuration can be beneficial, particularly when used with a vacuum suspension system.

For example, if a suspension sleeve (see, e.g., U.S. Pat. No. 6,406,499) is used by a below knee (BK) amputee, the lower end of the suspension sleeve will typically overlie the outside brim area of the prosthetic socket portion of an associated prosthesis, while the upper end of the suspension sleeve will overlie an exposed portion of the liner that is donned on the amputee's residual limb and extends above the brim of the socket. As such, imparting a liner of the present invention with a smooth surface along the area thereof that will extend beyond the brim of a prosthetic socket when donned allows for improved adhesion between the suspension sleeve and the underlying liner. Improved adhesion between a liner and suspension sleeve increases the likelihood that a vacuum can be maintained within a prosthetic socket when a vacuum suspension technique is employed.

Therefore, it can be understood that embodiments of a liner of the present invention may include a low-friction exterior surface with respect to that portion of the liner that will reside within a prosthetic socket when in use, and a smooth exterior surface with respect to that portion of the liner that will extend above the brim of the prosthetic socket when in use. With respect to the liner 85 shown in FIGS. 5a-5b, this is accomplished by a combination of the partial fabric exterior covering 105 and the exposed circumferential band 110 of underlying polymeric material.

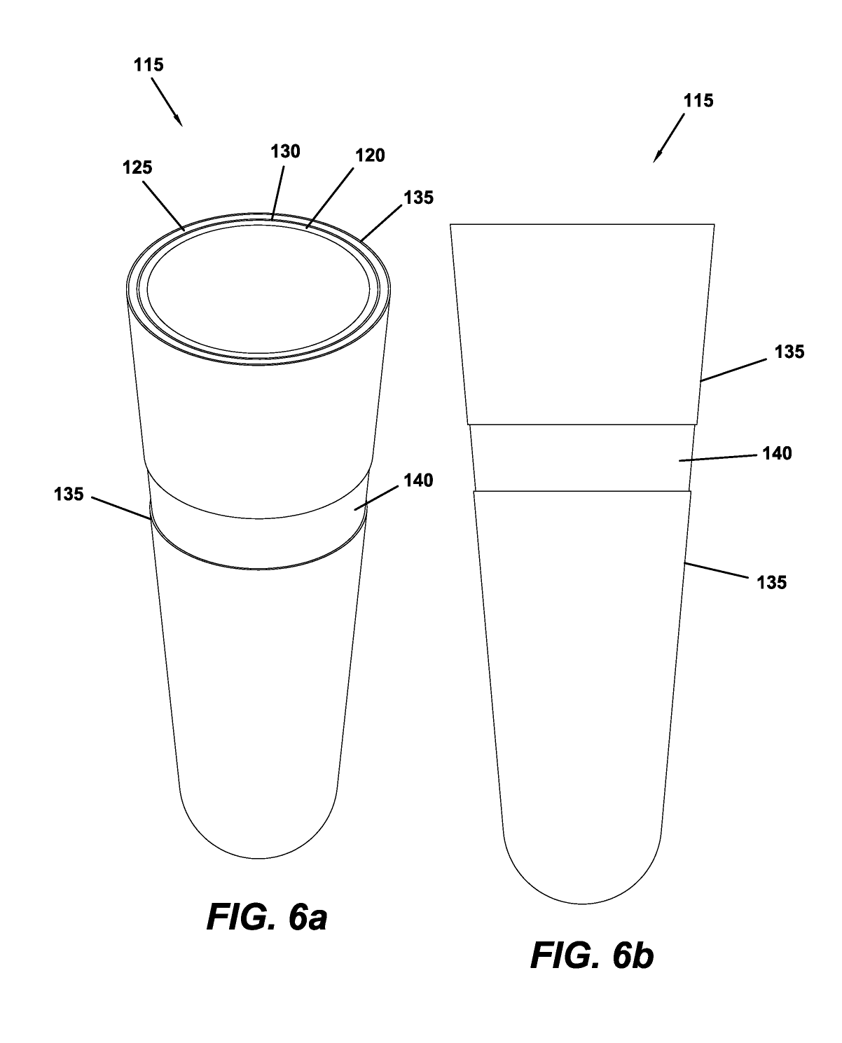

Another embodiment of a partially fabric-covered liner 115 of the present invention is depicted in FIGS. 6a-6b. The liner 115 again includes a softer inner polymeric material layer 120 that will abut the skin of a residual limb when donned, the inner polymeric material layer being surrounded by a harder outer polymeric material layer 125 that is separated therefrom by a fabric tying layer 130. Unlike the liner 85 of FIGS. 5a-5d, however, the outer surface of this exemplary liner 115 includes a fabric exterior covering 135 that is separated by an intermediary area of exposed polymeric material. Particularly, a fabric exterior covering 135 extends over substantially the entire exterior surface of the outer polymeric material layer 125, except for an intermediate portion 140 thereof where the underlying outer polymeric material layer is exposed.

In a similar manner to the exposed polymeric portion 110 of the liner 85 of FIGS. 5a-5b, the exposed intermediate portion 140 of this liner 115 may function to improve the performance of a vacuum suspension system. More specifically, during use, the exposed polymeric intermediate portion 140 of the liner may contact the inner wall of a prosthetic socket, producing a seal that will prevent (or at least greatly inhibit) the ability of air to leak from or be drawn into the prosthetic socket. The location of such an exposed polymeric portion may vary in other embodiments of a liner of the present invention, as may the number of such exposed areas provided.

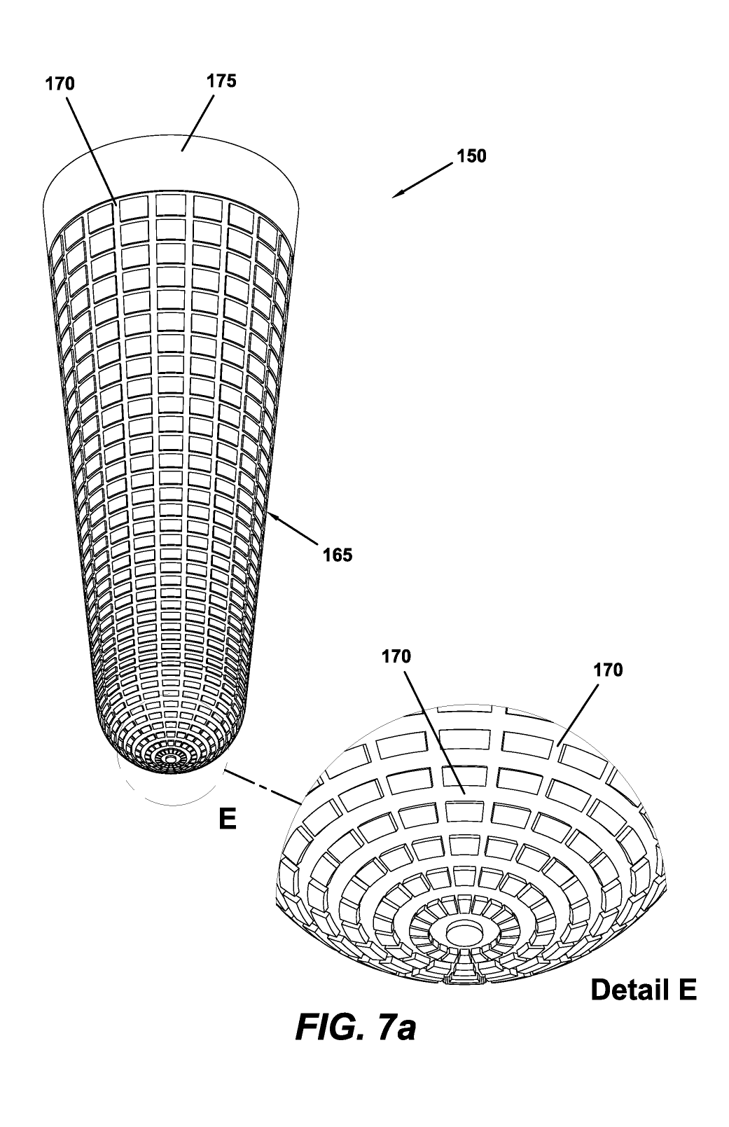

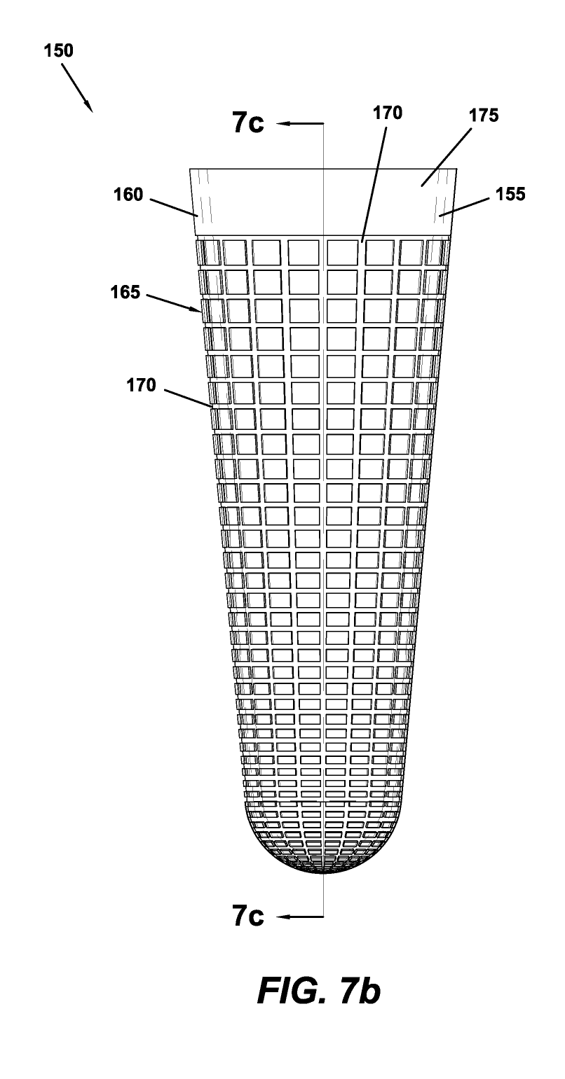

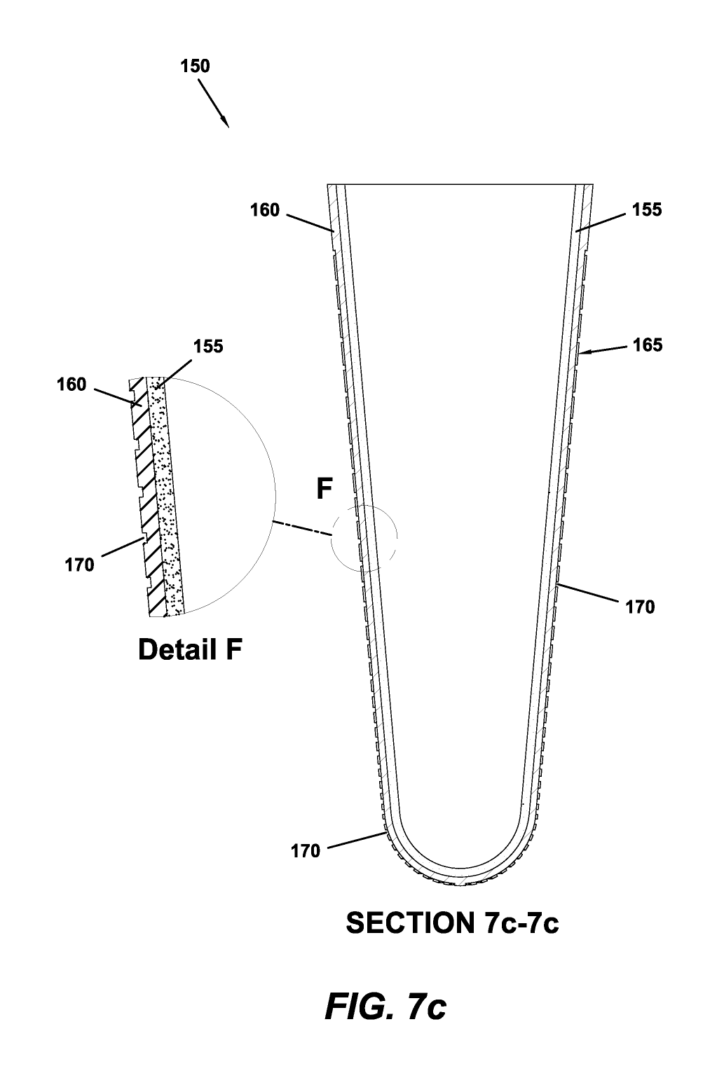

Another alternative exemplary embodiment of a liner of the present invention is disclosed in FIGS. 7a-7c. The liner 150 again includes a softer inner polymeric material layer 155 that will abut the skin of a residual limb when donned, the inner polymeric material layer being surrounded by a harder outer polymeric material layer 160. Unlike the other exemplary liners described and shown herein, however, the outer surface 165 of the outer polymeric material layer 160 of this exemplary liner 150 includes a multitude of molded wicking channels 170 that may function as previously described. As would be apparent to one skilled in the art, such wicking channels 170 may be produced during the liner molding process.

As shown in this particular embodiment, the wicking channels 170 cover substantially the entire outer surface 165 of the liner 150 (except for a portion 175 designed to extend beyond the rim of an associated prosthetic socket when the liner is donned). It is to be understood, however, that such wicking channels may cover only a portion(s) of a liner exterior in other embodiments. Similarly, the wicking channels may be of a shape and/or orientation that imparts a different pattern to a liner than the pattern shown in FIGS. 7a-7c.

The benefits of a liner of the present invention would be apparent to one skilled in the art from a reading of the foregoing description and a review of the related drawing figures. It would also be apparent to one skilled in the art that a number of different liner constructions may be created that will fall within the scope of the present invention. For example, a high-friction exterior surface like that shown in FIGS. 5a-5b may be used in conjunction with one or more high-friction exterior surfaces like that shown in FIGS. 6a-6b. Furthermore, while not specifically disclosed above or depicted in the drawing figures, nothing herein is to be interpreted as limiting a liner of the present invention to a construction having only two polymeric material layers. While it is believed that the use of only two polymeric material layers is advantageous from both an ease of manufacturing and cost saving perspective, the use of more than two such layers is nonetheless well within the scope of the present invention.

Similarly, while not specifically disclosed, nothing herein is to be interpreted as limiting a liner of the present invention to a construction having inner and outer polymeric material layers of dissimilar hardness. For example, it may be possible to manufacture a liner having inner and outer polymeric layers of substantially equivalent hardness while still providing an inner layer of offering superior comfort in comparison to the outer layer, and an outer layer of greater durability than that of the inner layer.

Therefore, while certain embodiments of the present invention are described in detail above, the scope of the invention is not to be considered limited by such disclosure, and modifications are possible without departing from the spirit of the invention as evidenced by the following claims:

What is claimed is:

1. A multi-layer prosthetic liner, comprising:
an inner polymeric material layer; and
an outer polymeric material layer surrounding said inner polymeric material layer, said outer polymeric material layer constructed of a material that is dissimilar to the material of said inner polymeric material layer.

2. The multi-layer prosthetic liner of claim 1, wherein said inner polymeric material layer is comprised of a thermoplastic elastomer gel.

3. The multi-layer prosthetic liner of claim 2, wherein said thermoplastic elastomer gel is a block copolymer.

4. The multi-layer prosthetic liner of claim 1, wherein said outer polymeric material layer is comprised of silicone, urethane, TPU, or TSPU.

5. The multi-layer prosthetic liner of claim 1, wherein said inner polymeric material layer has a hardness of between about 10-40 on the Shore 00 scale.

6. The multi-layer prosthetic liner of claim 1, wherein said outer polymeric material layer has a hardness of between about 30-70 on the Shore 00 scale.

7. The multi-layer prosthetic liner of claim 1, wherein said inner polymeric material layer and said outer polymeric material layer are joined by an adhesive.

8. The multi-layer prosthetic liner of claim 1, wherein said inner polymeric material layer and said outer polymeric material layer are joined by overmolding.

9. The multi-layer prosthetic liner of claim 1, wherein said inner polymeric material layer and said outer polymeric material layer are joined by an arrangement of interlocking elements.

10. The multi-layer prosthetic liner of claim 1, wherein an exterior surface of said outer polymeric material layer is wholly or partially covered with a low-friction material.

11. The multi-layer prosthetic liner of claim 10, wherein said material is fabric.

12. The multi-layer prosthetic liner of claim 1, wherein an exterior surface of said outer polymeric material layer is wholly or partially covered with a wicking material.

13. The multi-layer prosthetic liner of claim 12, wherein said material is a fabric.

14. The multi-layer prosthetic liner of claim 1, wherein said outer polymeric material layer is harder than said inner polymeric material layer.

15. The multi-layer prosthetic liner of claim 1, wherein said outer polymeric material layer is substantially impermeable to mineral oil.

16. The multi-layer prosthetic liner of claim 1, wherein said outer polymeric material layer has greater creep resistance, wear resistance, and/or puncture resistance than said inner polymeric material layer.

17. A multi-layer prosthetic liner, comprising:
an inner polymeric material layer;
an outer polymeric material layer surrounding said inner polymeric material layer, said outer polymeric material layer constructed of a material that is dissimilar to the material of said inner polymeric material layer; and
a tying layer disposed between said inner polymeric material layer and said outer polymeric material layer.

18. The multi-layer prosthetic liner of claim 17, wherein said tying layer is selected from the group consisting of a fabric, a fiber and a film.

19. The multi-layer prosthetic liner of claim 17, wherein said outer polymeric material layer is harder than said inner polymeric material layer.

20. The multi-layer prosthetic liner of claim 17, wherein said inner polymeric material layer has a hardness of between about 10-40 on the Shore 00 scale.

21. The multi-layer prosthetic liner of claim 17, wherein said outer polymeric material layer has a hardness of between about 30-70 on the Shore 00 scale.

22. The multi-layer prosthetic liner of claim 17, wherein said outer polymeric material layer is substantially impermeable to mineral oil.

23. The multi-layer prosthetic liner of claim 17, wherein said outer polymeric material layer has greater creep resistance, wear resistance, and/or puncture resistance than said inner polymeric material layer.
